(19) United States
(12) Patent Application Publication
(10) Pub. No.: US 2005/0165295 A1
(43) Pub. Date: Jul. 28, 2005

(54) Local magnetic resonance image quality by optimizing imaging frequency

(76) Inventors: Debiao Li, Naperville, IL (US); Vibhas Deshpande, Los Angeles, CA (US)

Correspondence Address: SCHIFF HARDIN LLP;Patent Department, 6600 Sears Tower, 233 South Wacker Drive, Chicago, IL, 60606, US

(21) Appl. No.: 10/984,101

(22) Filed: Nov. 9, 2004

Related U.S. Application Data
(60) Provisional application No. 60/538,825, filed on Jan. 23, 2004.

(51) Int. Cl. A61B 5/05

(52) U.S. Cl. 600/410; 600/413

(57) ABSTRACT

In a method and magnetic resonance (MR) imaging apparatus for reducing artifacts due to resonance frequency offsets in a diagnostic MR image, a number of MR scout images of a portion of a subject containing a region of interest (ROI) are generated respectively using different radio frequency (RF) excitation frequencies. Each of the MR scout images has an identifiable image quality in the ROI. The MR scout images are analyzed as to the image quality in the ROI to identify one of the MR scout images having the best image quality in the ROI. An MR diagnostic image is then generated of the portion of the subject containing the ROI, using the RF excitation frequency that was used to generate the MR scout image having the best image quality in the ROI.

### STATEMENT REGARDING FEDERALLY SPONSORED RESEARCH

The United States government has certain rights to this invention pursuant to Grant No. HL-38698 from the National Institutes of Health to Northwestern University.

### BACKGROUND OF THE INVENTION

1. Field of the Invention

The present invention relates generally to magnetic resonance imaging and, more particularly, to improving local magnetic resonance image quality by optimizing imaging frequency.

2. Description of the Prior Art

With improvements in gradient capabilities in recent years, true fast imaging with steady-state precession (true-FISP) has been successfully used in cardiac cine imaging and coronary artery imaging. In true-FISP, the zeroth moment of the gradients in each TR are zero so that transverse coherences are maintained in successive radio-frequency (RF) cycles. The resonance frequency offset then dominates the phase behavior of the spins and may cause image artifacts. One of the main sources for the off-resonance frequencies is B₀ field inhomogeneity. Another source is an incorrect setting of the synthesizer frequency (also referred to as the imaging frequency). Careful shimming can minimize these effects.

The correct estimation of the optimal imaging frequency is dependent on the field homogeneity. Achieving uniform fields by shim adjustments is particularly challenging in cardiac applications due to heart and respiratory motion, blood flow, chemical shift, and susceptibility variations at air-tissue interfaces. When phase is used to estimate the field distortions, anatomic motion and blood flow may cause errors. Therefore, it is difficult to develop a general shim solution for continually changing heart position and geometry. Suboptimal shimming then gives rise to field inhomogeneity and variations in resonant frequency. Jaffer et al. (A Method to Improve the Homogeneity of the Heart in vivo, Magn. Resonance in Med., Vol. 36 (1996) pp. 375-383) reported that a peak-to-peak gradient of 62 Hz may be present across the heart at 1.5 T. Also, in a study by Reeder et al. (In vivo Measurement of T2* and Field Inhomogeneity Maps in the Human Heart at 1.5 T, Magn. Resonance in Med., Vol. 39, (1998) pp. 988-998) frequency offsets on the order of 100 Hz were found in the vicinity of the cardiac veins. Therefore, no matter what imaging frequency is selected, certain spins will have resonance offsets in the heart. In addition, the frequency estimated by adjustment routines may not be optimal for the heart due to the different volumes used for frequency adjustment and imaging, and/or the presence of tissues other than the heart (chest wall, liver, etc.) in the prescribed adjustment volume when a large field inhomogeneity is present.

The presence of resonance frequency offsets often causes artifacts in images acquired with true fast imaging with steady-state precession (true-FISP). One source of resonance offsets is a suboptimal setting of the synthesizer frequency. A good quality image requires that “imaging” and “resonance” frequencies are well matched. The current technique is to estimate the average resonance frequency across the entire imaging scene, then to try to match the imaging frequency to the average resonance frequency. Local resonance frequencies vary from their average across a scene of view, so matching to the average can mean mismatches and blurred image in local regions.

Fat saturation using a chemical shift prepulse is also sensitive to field inhomogeneities and the water frequency selected for imaging. The fat saturation pulse assumes a chemical shift of approximately 3.2 parts per million (ppm) from the resonance frequency of water. Since field distortions can alter the fat frequency, the suppression may be compromised if a fixed frequency offset is used when the shimming or the selected water frequency is suboptimal. If the optimal frequency offset for fat is determined for the volume of interest (VOI), the fat suppression can be improved.

The current technique that attempts to set imaging frequency to average resonance frequency is to shim the magnets, but shimming can result in large mismatches for dynamic applications. Heart motion, respiratory motion, and blood flow in cardiac imaging are just some of the factors that create a highly dynamic imaging scene that is not conducive to shimming. Shimming can take considerable time, and the shims need to be modified for each patient. Shimming only matches imaging frequency with the average resonance frequency, and not with the local resonance frequency. This can lead to imaging artifacts in the region of greatest interest to a physician for a given patient.

In magnetic resonance imaging, the strength of the applied static magnetic field (main or basic field) determines the oscillating frequency (the resonance frequency) of the tiny internal magnetic field created by human tissue. The correct mapping of the human internal organ anatomy relies on the exact matching of the frequency of the applied oscillating magnetic field (radiofrequency or RF field) with the resonance frequency of the human tissue. In practice, the main field is not uniform across the imaging plane, resulting in variable resonance frequencies in different areas of the imaging plane. It is necessary, however, for a uniform core frequency of the RF field (imaging frequency) to be applied over the entire imaging volume. The imaging frequency usually is determined automatically by the imaging system to match the average resonance frequency of the tissues in the entire imaging volume, which may not match the resonance frequency of a particular region. Therefore, there are always mismatches between the resonance frequencies of the tissues and the imaging frequency in certain parts of the imaging plane. This mismatch usually results in signal intensity variations and artifacts in images. Efforts have been made to improve the uniformity of the main field, however, field inhomegeneity always exists in practical imaging conditions, which may result in image artifacts, particularly for fast imaging with steady state precession, a type of imaging technique commonly used in magnetic resonance imaging of the heart in recent years.

### SUMMARY OF THE INVENTION

The inventive method and apparatus overcome the limitations of the prior art by determining the imaging frequency that best matches the local resonance frequency for the region of interest. A simple scouting method—a “prescan”—is used to estimate the optimal imaging frequency for the local area of interest. The prescan can be taken in a single breath hold—an important requirement in cardiac imaging. With the invention, a single prescan acquires multiple images at multiple frequencies that can be compared to find the optimum.

In accordance with the invention, if apparent off-resonance artifacts are present in true-FISP images, changing the automatically adjusted imaging frequency can improve image quality in some cases. Coronary artery imaging using 3D segmented true-FISP was performed to demonstrate these effects. A prescan simulating different water imaging frequencies was designed and the quality of images acquired from the scan was examined visually to determine the optimal imaging frequency. A similar sequence with variable fat saturation pulse frequency offsets was developed to optimize the fat saturation pulse frequency. Volunteer studies were conducted to compare the image quality with both the automatically adjusted and manually determined imaging frequencies and fat saturation frequency offsets.

The present invention provides an imaging frequency shifting concept and method to improve image quality of magnetic resonance imaging. The invention provides fast magnetic resonance imaging of the heart and other organs to obtain high resolution and clear images.

In accordance with the invention, the imaging frequency is shifted to match the local resonance frequency of the region of interest. This optimizes the image quality in the region of interest. The optimal imaging frequency for a particular region of interest is identified by acquiring pre-scans. Multiple scout images are collected with different imaging frequencies before the actual imaging scan. The scout scans can be acquired from a single slice with low spatial resolution, thus are much faster than the actual imaging scan which usually covers multiple slices and requires relatively high spatial resolution. Image quality of the scout scan is then examined. The imaging frequency that gives the best image quality is used for the actual imaging scan. This process can be repeated to fine-tune the imaging frequency.

Poor image quality resulting from main field inhomogeneities is a major obstacle for magnetic resonance imaging, particularly in the heart. The method and apparatus of the present invention substantially improve the image quality.

As is stated above, the presence of resonance frequency offsets often causes artifacts in images acquired with true fast imaging with steady-state precession (true-FISP). One source of resonance offsets is a suboptimal setting of the synthesizer frequency. Shifting the synthesizer frequency minimizes the off-resonance related image artifacts in true-FISP. A simple scouting method estimates the optimal synthesizer frequency for the volume of interest (VOI). To improve fat suppression, a similar scouting method determines the optimal frequency offset for the fat saturation pulse. Coronary artery imaging performed in healthy subjects using a 3D true-FISP sequence validates the effectiveness of the frequency corrections. Substantial reduction in image artifacts and improvement in fat suppression were observed by using the water and fat frequencies estimated by the scouting scans. Frequency shifting is a useful and practical method for improving coronary artery imaging using true-FISP.

### DESCRIPTION OF THE DRAWINGS

FIG. 1 schematically illustrates a frequency spectrum acquired in a subject to compare improper frequency adjustment with the automatic frequency adjustment in accordance with the invention.

FIG. 2 schematically illustrates a frequency scouting 2D true-FISP sequence suitable for use in accordance with the present invention.

FIG. 3 shows images acquired at various frequency offsets using the frequency scouting sequence of FIG. 2.

FIGS. 4A and 4B respectively show images of the LAD acquired before and after inventive frequency correction.

FIG. 5 shows fat frequency scout images (top row) and images acquired using the 3D true-FISP sequence before and after correction of the fat saturation pulse frequency offset (bottom row), in accordance with the invention.

FIGS. 6A and 6B respectively show images of the RCA acquired before and after inventive correction of the frequency offset of the fat saturation pulse.

FIG. 7 is a schematic block diagram showing the basic components of an apparatus constructed and operating in accordance with the principles of the present invention.

### DESCRIPTION OF THE PREFERRED EMBODIMENTS

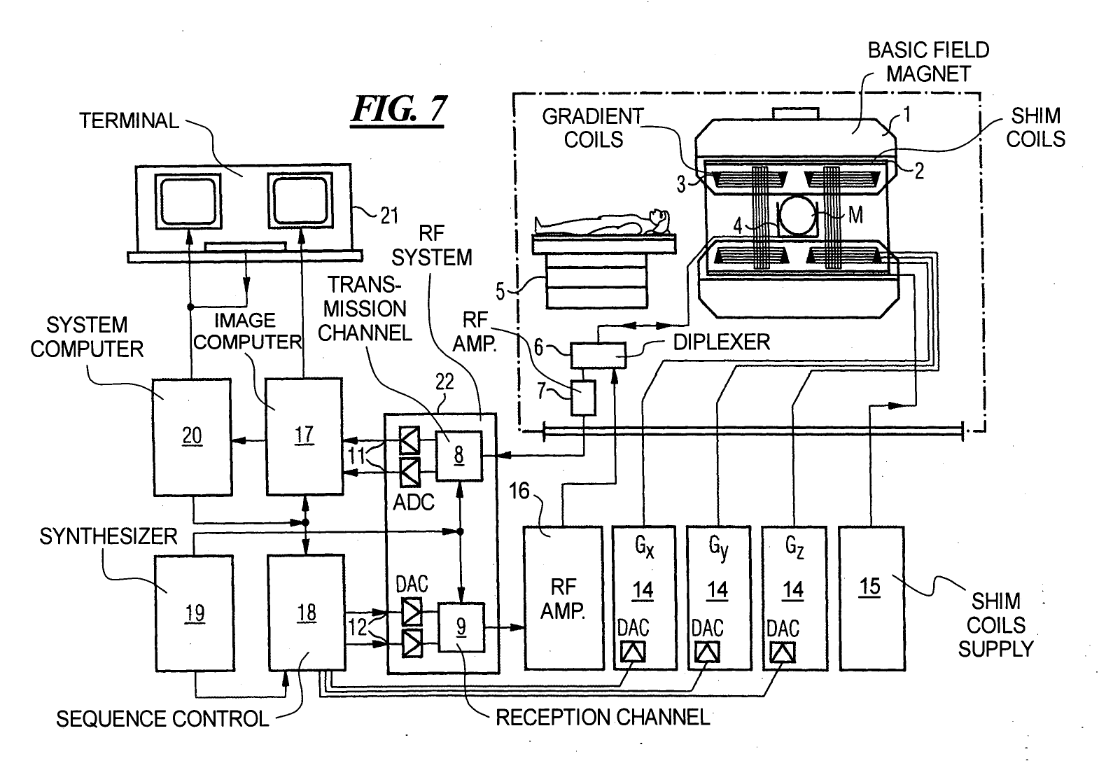

FIG. 7 schematically illustrates a magnetic resonance imaging (tomography) apparatus for generating a nuclear magnetic image of a subject according to the present invention. The components of the nuclear magnetic resonance tomography apparatus correspond to those of a conventional tomography apparatus, but it is controlled according to the invention. A basic field magnet 1 generates a time-constant, intense magnetic field for polarization (alignment) of the nuclear spins in the examination region of a subject such as, for example, a part of a human body to be examined. The high homogeneity of the basic magnetic field required for the nuclear magnetic resonance measurement is defined in a spherical measurement volume M in which the part of the human body to be examined is introduced. For supporting the homogeneity demands and, in particular, for eliminating time-invariable influences, shim plates of ferromagnetic material are attached at suitable locations. Time-variable influences are eliminated by shim coils 2 that are driven by a shim power supply 15.

A cylindrical gradient coil system 3 is built into the basic field magnet 1, the system 3 being composed of three sub-windings. Each sub-winding is supplied with current by an amplifier 14 for generating a linear gradient field in the respective directions of a Cartesian coordinate system. The first subwinding of the gradient field system 3 generates a gradient Gx in the x-direction, the second sub-winding generates a gradient Gy in the y-direction, and the third sub-winding generates a gradient Gz in the z-direction. Each amplifier 14 has a digital-to-analog converter DAC that is driven by a sequence control 18 for the time-controlled generation of gradient pulses.

A radio-frequency antenna 4 is situated within the gradient field system 3. The antenna 4 converts the radio-frequency pulses emitted by a radio-frequency power amplifier into an alternating magnetic field for exciting the nuclei and aligning the nuclear spins of the subject under examination, or of a region of the subject under examination. The radio-frequency antenna 4 is composed of one or more RF transmission coils and a number of RF reception coils in the form of an arrangement (preferably linear) of component coils. The alternating field proceeding from the precessing nuclear spins, i.e. the nuclear spin echo signals produced as a rule by a pulse sequence composed of one or more radio-frequency pulses and one or more gradient pulses, is also converted into a voltage by the RF reception coils of the radio-frequency antenna 4, this voltage being supplied via an amplifier 7 to a radio-frequency reception channel 8 of a radio-frequency system 22. The radio-frequency system 22 also has a transmission channel 9 wherein the radio-frequency pulses are generated for exciting magnetic nuclear resonance. The respective radio-frequency pulses are digitally presented as a sequence of complex numbers on the basis of a pulse sequence in the sequence control 18 prescribed by the system computer 20. This number sequence—as a real part and an imaginary part—is supplied via respective inputs 12 to a digital-to-analog converter DAC in the radio-frequency system 22 and is supplied from there to a transmission channel 9. In the transmission channel 9, the pulse sequences are modulated onto a radio-frequency carrier signal having a basic frequency corresponding to the resonant frequency of the nuclear spins in the measurement volume.

The switching from transmission mode to reception mode ensues via a transmission/reception diplexer 6. The RF transmission coil of the radio-frequency antenna 4 radiates the radio-frequency pulses, based on signals from a radio-frequency amplifier 16, for excitation of the nuclear spins into the measurement volume M and samples the resulting echo signals via the RF reception coils. The acquired nuclear magnetic resonance signals are phase-sensitively demodulated in the reception channel 8 of the radio-frequency system 22 and are converted via respective analog-to-digital converters ADC into the real part and the imaginary part of the measured signal, which are respectively supplied to outputs 11. An image computer 17 reconstructs an image from the measured data acquired in this way (including, when appropriately programmed or instructed, organizing the data in accordance with the invention). Administration of the measured data, the image data and the control programs ensues via the system computer 20. On the basis of control programs, the sequence control 18 monitors the generation of the respectively desired pulse sequences and the corresponding sampling of k-space. In particular, the sequence control 18 controls the tined switching of the gradients, the emission of the radio-frequency pulses with defined phase and amplitude, as well as the reception of the nuclear magnetic resonance signals in accordance with the invention according to a control program designed to implement the inventive method. The timing signals for the radio-frequency system 22 and the sequence control 18 is made available by a synthesizer 19. The selection of corresponding control programs for generating a nuclear magnetic resonance image as well as the presentation of the generated nuclear magnetic resonance image ensues via a terminal 21 that has a keyboard as well as one or more picture screens.

Before an image is acquired of a subject, the resonance frequency of the proton on which the image will be based is first determined. Frequency adjustments are performed on a scanner by generating a frequency spectrum from the free induction decay (FID) signal acquired from a user-defined volume. The peak frequency in this spectrum is used as the synthesizer frequency. Ideally, the volume used for the adjustment should correspond to the volume of interest (VOI). However, in situations where the VOI is very small (such as in coronary artery imaging), the adjustment volume may need to be increased in the interest of frequency adjustment signal-to-noise ratio (SNR). In this case, the peak frequency in the spectrum may not represent the optimal resonant frequency for the VOI if field inhomogeneities are present, which in turn may cause off-resonance artifacts. For example, for coronary artery imaging in a coronal plane, the liver signal may dominate during the frequency adjustment if it is present in the adjustment volume.

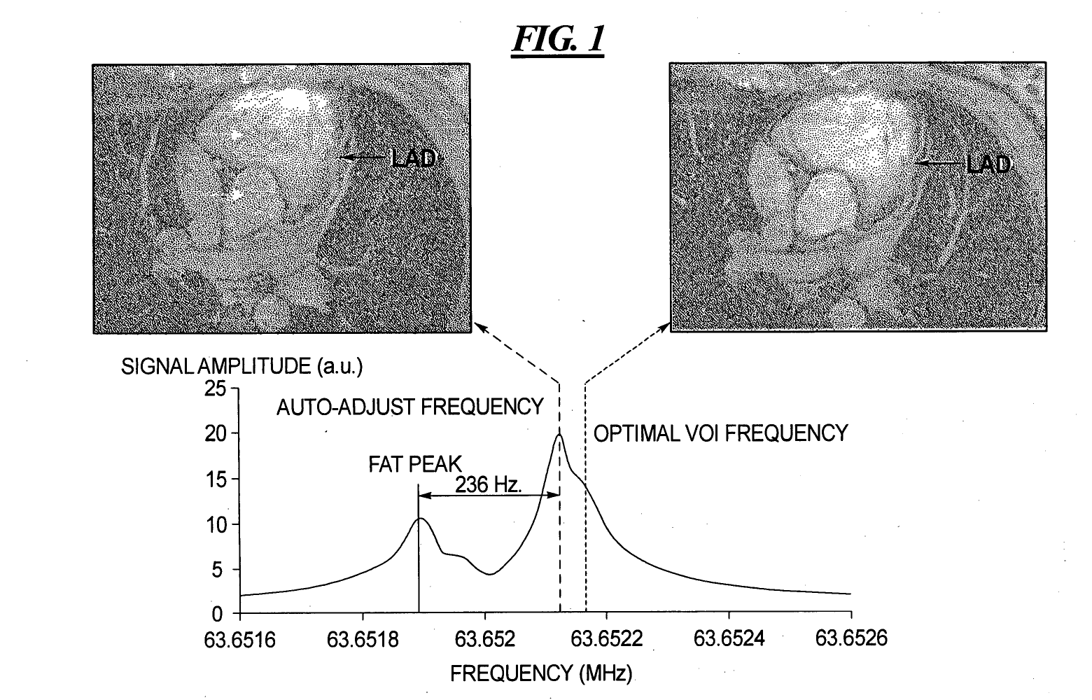

An example of improper adjustment of the frequency is shown in FIG. 1. The frequency spectrum was acquired in a subject using the standard frequency adjustment routine available on the apparatus of FIG. 7. A whole-body coil was used as the transmitter and receiver. The adjustment volume was the same as the imaging volume, which was localized for scanning the left anterior descending coronary artery (LAD). It can be seen that the water peak is broad, with a full width at half maximum of 91 Hz. The auto-adjusted frequency was found to be 63.652124 MHz, whereas the optimal frequency determined for the VOI was found to be 63.652164 MHz—a difference of 40 Hz. The images acquired at these frequencies are shown. It can be seen that the artifacts (dashed arrows) are reduced and the delineation of the LAD is better in the image acquired at the optimized frequency.

Image artifacts will be reduced in the coronary artery if the synthesizer frequency is shifted to the frequency that is optimal for the VOI containing the coronary artery, even though it may not be the peak frequency for the entire adjustment volume. Resonance offset artifacts are then likely to appear in other regions of the field of view (FOV). Such artifacts may be permissible as long as they do not interfere with the coronary artery.

Simulating Frequency Offsets Using RF Phase

Since true-FISP is sensitive to off-resonance, the optimal resonance frequency can be determined by acquiring images at various frequencies and visually examining the depiction of the coronary arteries. However, searching for the optimal frequency by changing it iteratively for each scan can be a time-consuming process. According to the invention, this disadvantage is overcome by use of a single prescan method for acquiring images at different resonant frequencies.

One technique for practical implementation is to use RF transmitter and ADC phase shifts to simulate frequency offsets. Let f be the frequency of the rotating frame, which is defined by the synthesizer frequency setting. To achieve frequency variations, the phase of every RF pulse is incremented by φ°, i.e., the phase of the first RF pulse is incremented by φ, the phase of the second pulse is incremented by 2φ, etc. The angular frequency of the rotating frame is then altered to (f+φ/TR). Therefore, each φ represents a unique frequency offset equal to φ/TR. To investigate the spin system from the new rotating frame, the phase of the ADC is also incremented by φ in each cycle. One way to change the imaging frequency is to change the frequency setting of the synthesizer.

Frequency Scouting Sequence Design

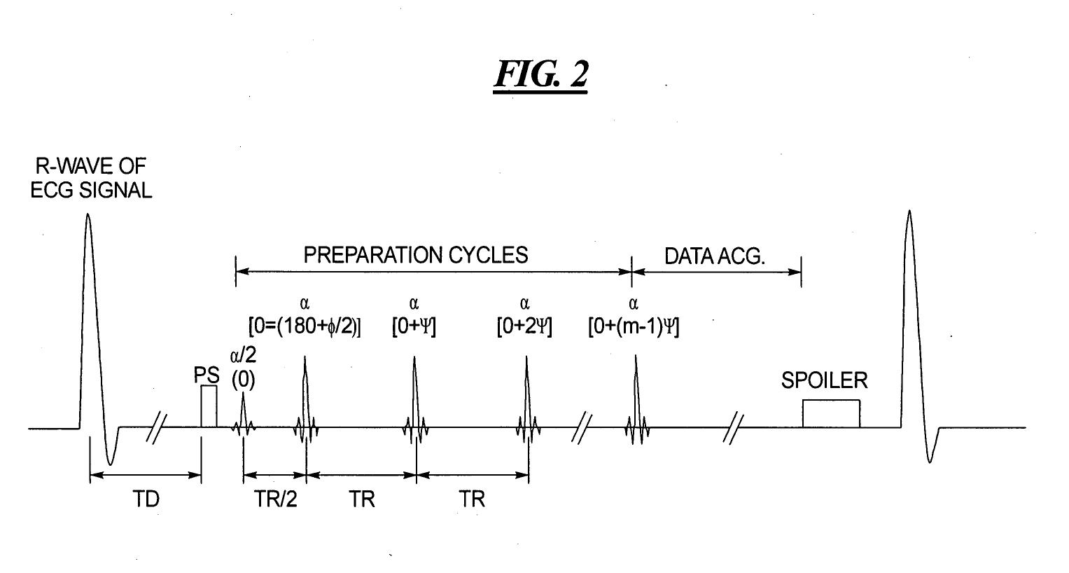

The two-dimensional (2D), segmented true-FISP proton frequency scouting sequence is shown in FIG. 2.

After an appropriate trigger delay (TD) time, a spectrally selective fat saturation (FS) pulse is applied, followed by an α/2 preparation pulse. This is followed by 20 preparation cycles with flip angle α and then the data acquisition cycles, also with flip angle α. The phases of the RF excitations are shown on top of the pulses in brackets. The ADC (not shown) phase in each data acquisition cycle is equal to the preceding RF pulse phase. The phase offset φ is added to the successive RF pulses and the ADCs to simulate different synthesizer frequencies. Notice that the phase added to the first α preparation cycle is φ/2 because the interval between the α/2 prepulse and the first α pulse is TR/2. Multiple measurements are performed in a single breath-hold. In successive measurements, φ is changed iteratively to simulate different frequencies, m=excitation number excluding the α/2 pulse; ψ=180+φ.

The sequence is designed so as to emulate the acquisition scheme of the 3D coronary artery imaging sequence. Electrocardiographic (ECG) triggering is used, and a fat saturation pulse followed by an α/2 prepulse (α=data acquisition flip angle) and 20 constant flip angle preparation cycles are applied before data acquisition in each cardiac cycle. The data are acquired centrically in the phase-encoding direction. Three cardiac cycles are required to acquire one image. Phase alternation (phase incremented by 180°) is implemented in successive RF cycles. To simulate frequency offsets, a phase offset φ is added to the successive RF pulses and ADC, as described above. Therefore, the total phase increment for successive RF pulses and ADC is the sum of the phase alternation and the desired frequency offset; e.g., to generate a frequency offset of 50 Hz at a TR of 3.6 ms, the total phase increment is (180+64.8)°. Multiple measurements are performed at the same slice position within a single breath-hold, each measurement with a different phase offset φ. Thus, each measurement then represents an image acquired at a different frequency offset from the synthesizer frequency. The images are examined visually and the frequency corresponding to the one with the least artifacts is estimated as the optimal imaging frequency.

To find the optimal fat frequency, the same sequence is used with φ set to zero. The frequency offset of the fat saturation pulse from the synthesizer frequency is varied in each measurement. The measurement that shows the best fat suppression in the image indicates the optimal frequency offset to be used for the fat saturation pulse.

TABLE 1
Summary of the Optimized Proton and Fat Frequency Shifts From the
Synthesizer Frequency for the Subjects Scanned in the Study

| Subject | Proton | | |
|---|---|---|---|
| number | frequency shift | | Fat Frequency shift |
| 1 | −40 | Hz | * |
| 2 | −160 | Hz | * |
| 3 | +30 | Hz | * |
| 4 | +30 | Hz | −290 Hz |
| 5 | +40 | Hz | −250 Hz |
| 6 | −30 | Hz | −210 Hz |
| 7 | −30 | Hz | −260 Hz |
| 8 | 0 | Hz | −250 Hz |
| 9 | 0 | Hz | −290 Hz |
| 10 | 0 | Hz | −270 Hz |
| 11 | 0 | Hz | −190 Hz |

* No fat frequency scouting was performed for the subject

Only those subjects where a frequency shift (either water or fat) was necessary are shown. The proton frequency was adjusted first if it was found to be suboptimal. Following this adjustment, the fat frequency offset was optimized with respect to the new synthesizer frequency. In six out of the 14 subjects scanned, the proton frequency was shifted. Of the 11 subjects scanned for optimizing the fat saturation pulse frequency offsets, the frequency offset was found suboptimal in seven cases.

Coronary Artery Imaging Experiments

All imaging experiments were performed on a 1.5 T whole-body MR scanner (Siemens Magnetom Sonata, Erlangen, Germany) with a high-performance gradient subsystem (maximum amplitude=40 mT/m, maximum slew rate=200 mT/m/ms). Coronary artery imaging was performed in 14 healthy volunteers (including nine males and four females, 26-53 years old, mean age 37.5 years) using an ECG-triggered, breath-hold, segmented, 3D true-FISP sequence. A linear flip angle series preparation was used to reduce the sensitivity of the signal to resonance offsets. Asymmetric sampling was implemented to shorten the TR and TE. Images using a low-resolution localizer scan were first acquired using the above sequence. A three-point tool was used to prescribe the imaging planes for the LAD and right coronary artery (RCA) based on the low-resolution images. High-resolution true-FISP scans were then performed along these orientations.

If apparent off-resonance artifacts were observed in the images, the frequency scout sequence was run in the same orientation as the high-resolution scan. A coarse frequency scout scan was first acquired with effective frequency offsets of −80 Hz to +120 Hz, in steps of 40 Hz. Because of the limit of the breath-hold time, only six offset frequencies were tested in a single scout scan. This was followed by a finer adjustment in steps of 10 Hz in the vicinity of the optimal frequency determined by the coarse adjustment. If the scout sequence indicated that a shift in frequency was necessary, the synthesizer frequency was changed and the 3D high-resolution scan was repeated at the modified frequency. Next, if the fat suppression was found to be suboptimal, the sequence for scouting the fat frequencies was performed. Again, if the optimal offset was found to be other than −210 Hz, the high-resolution scan was repeated with the fat saturation pulse applied at a frequency offset that was determined from the scout.

The imaging parameters for the frequency scout sequence were as follows: TR/TE=3.6/1.8 ms, flip angle=70°, readout bandwidth=980 Hz/pixel, FOV=(160-175)×300 mm², data acquisition matrix size=(75-105) ×256, slice thickness=10 mm, number of measurements=6, breath-hold time=18 cardiac cycles. Depending on the heart rate of the subject, the number of lines acquired per cardiac cycle varied from 25 to 35. The imaging parameters for the high-resolution 3D scans were as follows: TR/TE=3.55/1.44 ms, flip angle=70°, readout bandwidth=810 Hz/pixel, FOV=(160-175) ×380 mm², data acquisition matrix size=(100-140)×512, lines per cardiac cycle=25-35, and breath-hold time=24 cardiac cycles. Four cardiac cycles were required to acquire one kₓ-k_{y} plane before the partition-encoding gradient was incremented. The number of partitions acquired was 6, which was then sinc interpolated to 12. The partition thickness was 1.5 mm after interpolation, and the resultant coverage was 18 mm for each slab. A phased-array two-channel coil was used as the receiver for the volunteer studies.

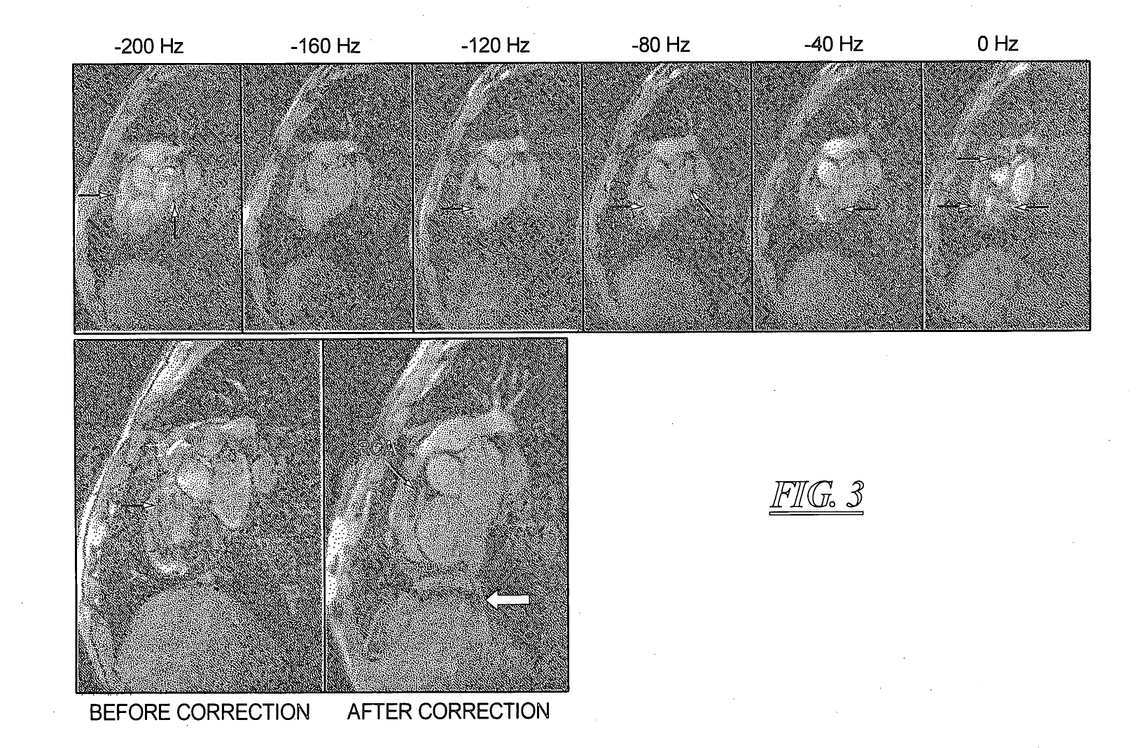

The automatically adjusted imaging frequency was found to be suboptimal in six of the 14 subjects scanned. Two scout scans (coarse and then fine tuning) were then acquired to determine the optimal frequency corresponding to each coronary artery image orientation. The frequency shifts in each case are given in Table 1. An example of the frequency scout images, and comparisons of the images acquired before and after the frequency correction are shown in FIG. 3.

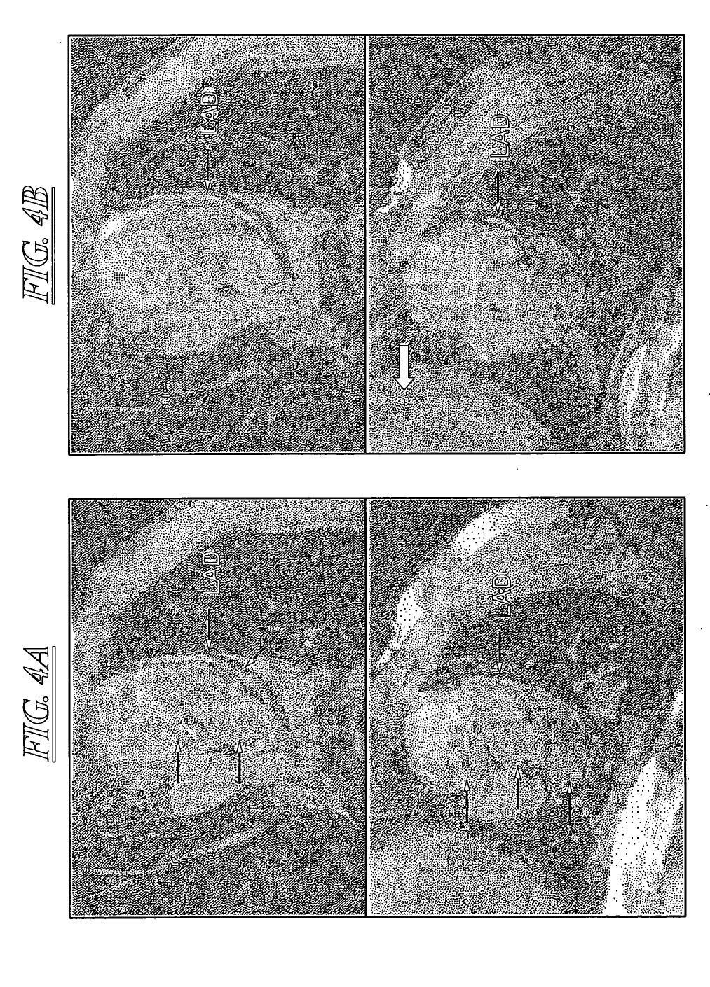

FIG. 3 shows images acquired at various frequency offsets using the frequency scouting sequence (top row), and images acquired using the 3D true-FISP sequence before and after inventive correction of the imaging frequency (bottom row). The 3D true-FISP image at the automatically adjusted frequency (before correction) shows substantial artifacts (dashed arrows). The frequency scout images acquired in a range of −200 Hz to 0 Hz indicate that the optimal frequency offset is −160 Hz because the blood pool is relatively uniform in the corresponding image. With a shift of −160 Hz in the synthesizer frequency, considerable reduction in the artifacts is observed in the 3D true-FISP image (after correction) and the RCA is now clearly visible. After altering the imaging frequency, the image artifacts were substantially reduced and the RCA was clearly visualized. It can be seen that there was no artifact in the liver at the original frequency, but an artifact was introduced after the frequency was shifted. However, this artifact did not affect the delineation of the coronary artery. More examples of images before and after frequency correction are shown in FIGS. 4A and 4B. In both cases, the depiction of the coronary artery was improved after the frequency was corrected. Again, in one of the image sets, artifacts were seen in the liver after the frequency was shifted.

FIGS. 4A and 4B respectively show images of the LAD acquired before and after inventive frequency correction. The frequency was corrected by +40 Hz for the images in the top row and by +70 Hz for the images in the bottom row. In both cases, the image artifacts (dashed arrows) were substantially reduced after the frequency correction. It can be seen that image artifacts appear in the liver (block arrow) in the corrected image of the bottom row.

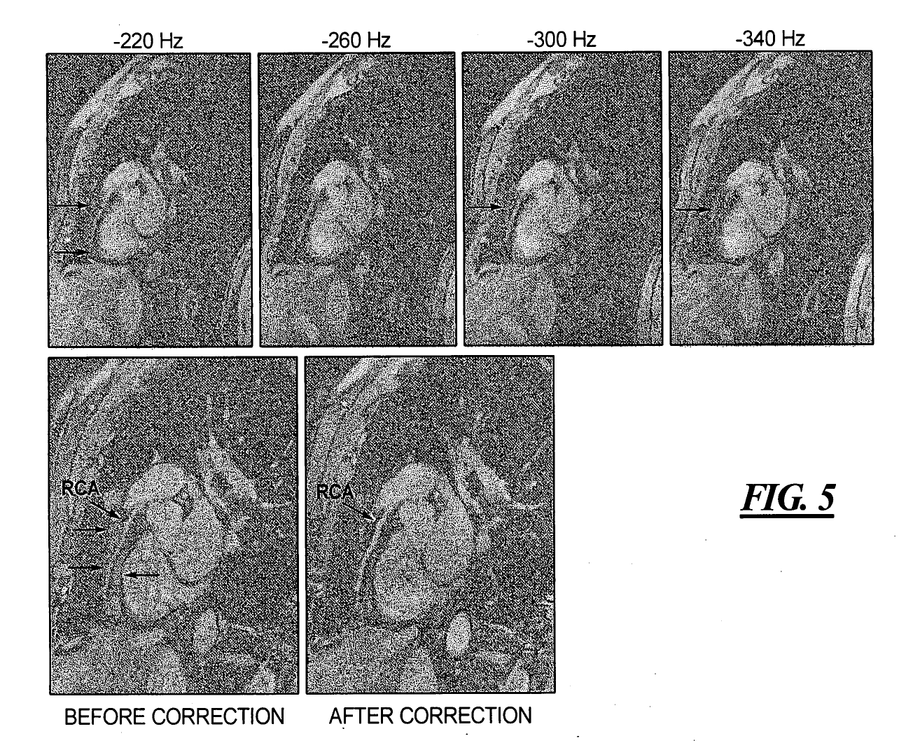

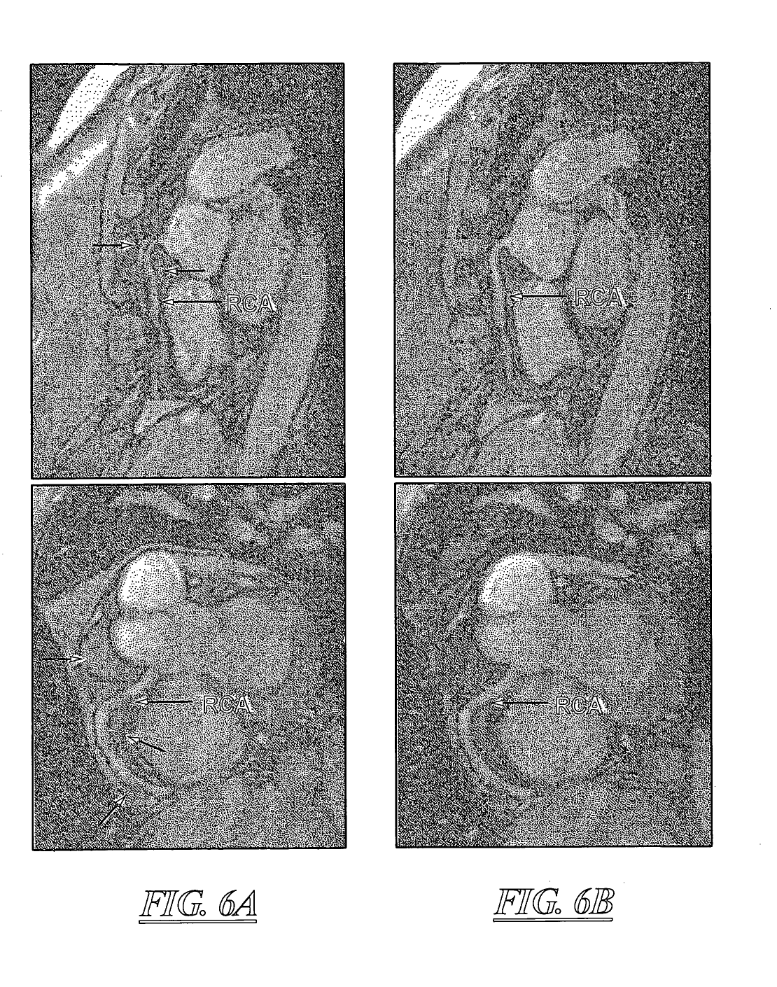

In seven of 11 volunteers scanned for fat frequency shifts, the routinely used −210 Hz fat saturation frequency offset was found to be suboptimal. The optimized fat saturation pulse frequency offsets from the synthesizer frequency for each of the subjects are specified in Table 1 above. An example of the scout images and images acquired before and after altering the fat saturation frequency is shown in FIG. 5. FIG. 5 shows fat frequency scout images (top row) and images acquired using the 3D true-FISP sequence before and after correction of the fat saturation pulse frequency offset (bottom row). The 3D true-FISP image with the fat saturation pulse at −210 Hz offset (before correction) shows that the fat signal surrounding the RCA is not suppressed (dashed arrows). The scout images acquired with the variation in the fat saturation pulse frequency offset indicate that the best suppression is obtained with a frequency offset of −260 Hz for the chemical shift pulse. When the fat saturation pulse frequency offset is set to −260 Hz, the 3D true-FISP image (after correction) shows better suppression of the fat signal. It can be seen that the phase cancellation of the signal at the boundaries of the RCA in the pre-correction image is reduced in the image acquired after correction. The image acquired after correction shows an improved delineation of the coronary artery, and reduced phase cancellation artifacts at the blood-fat boundaries. Other examples of images acquired before and after the fat saturation pulse offset correction are shown in FIGS. 6A and 6B. In both cases the fat saturation and coronary artery definition was improved by the correction. FIGS. 6A and 6B respectively show images of the RCA acquired before and after correction of the frequency offset of the fat saturation pulse. Since the fat signal is not suppressed (dashed arrows) in the images in FIG. 6A, phase cancellation artifacts are visible at the blood-fat boundaries. The optimized frequency offset of the fat saturation pulse from the synthesizer frequency was −190 Hz for the corrected image in the top row, and −290 Hz for the corrected image in the bottom row. The images of FIG. 6B show that fat suppression is improved in both cases after the correction is made.

The sensitivity of the signal to resonance offsets remains one of the major problems in true-FISP imaging. When true-FISP is used for coronary artery imaging, resonance offset artifacts can often be reduced in the VOI by shifting the imaging frequency. The inaccuracies in the automatic frequency adjustments may arise due to the presence of field inhomogeneities. Achieving a homogeneous field by shimming is challenging in the heart, for the various reasons noted above. If phase-sensitive techniques are used for shim adjustments, anatomic motion is one of the hindrances to estimating a solution. Another problem caused by motion is the constant changes in the prescribed adjustment volume. A logical extension, therefore, is to use respiratory and ECG-gated shim adjustments, but this significantly increases the time required for shimming. Another option for overcoming the shim adjustment problems due to motion is to use chemical shift imaging which can provide a substantial reduction in the standard deviation of the proton frequency associated with shimming in cardiac applications. Also, respiratory-gated and/or ECG-gated frequency adjustment methods can be used.

Obvious off-resonance artifacts are observed in the coronary artery true-FISP images if the shimming and/or frequency adjustment is suboptimal. Shimming may not be a reliable solution in these situations because of the problems mentioned above. Despite suboptimal shimming, however, shifting the frequency to that which is optimal for the VOI reduces image artifacts in most cases. This was demonstrated in the imaging experiments in which no shimming was performed, and the image artifacts were reduced in the coronary artery in each case with only a frequency shift. Artifacts were induced in the liver in several volunteers after the frequency shift because the imaging frequency was shifted away from the resonant frequency of the liver. These artifacts did not affect the depiction of the coronary arteries.

Searching for the optimal frequency by changing the synthesizer frequency for each scan requires separate scans and is time-consuming. Therefore, the method and apparatus of the invention implement a prescan to estimate the optimal imaging frequency in a single breath-hold. The images from the prescan are examined visually to determine the optimal imaging frequency. It would also be possible, depending on the image quality, to automatically electronically identify the pre-scan image having the heat image quality, such as by automatic noise analysis or pattern recognition or other suitable techniques. In almost half of the volunteers scanned in this study, the automatically adjusted water frequency was found to be suboptimal. The prescan reliably estimated an optimal frequency, which improved the depiction of the coronary arteries in each case. As fat saturation is also sensitive to the field inhomogeneities and synthesizer frequency setting, another prescan can be performed in accordance with the invention to optimize the fat saturation pulse frequency offsets from the synthesizer frequency. When the 3D true-FISP sequence is used, the optimal frequency offset of the fat saturation pulse from the synthesizer frequency (or the optimized water frequency) is not equal to −210 Hz in most subjects, and can vary in each case. The prescan again provides reliable estimates of the fat saturation pulse frequency offset.

Although the frequency scout scan is acquired in a 2D slice while the coronary artery images are acquired in a 3D slab, the optimal frequencies of the two scans are very similar because the thicknesses of the 2D slice and 3D slab are similar [10 mm and 18 mm, respectively) and the field changes and the corresponding variations in image quality are relatively slow. Improved coronary artery delineation was observed in all subjects that required frequency shifting based on scout scans.

In summary, although the performance of true-FISP is highly sensitive to off-resonance, adjustments in frequency improve the image quality of a particular VOI. This is especially useful in the heart, where shimming is difficult. Results in the studies discussed herein show that the image artifacts in the VOI are reduced in all of the cases when the frequency is shifted to the optimal frequency determined by the frequency scout scan. Frequency scouting sequences as described provide a fast, easy, and reliable method to optimize the proton and fat frequencies for coronary artery imaging using 3D true-FISP. Such adjustments may benefit other frequency-sensitive techniques as well, such as projection reconstruction and spiral imaging.

The above description of the preferred embodiment of the present invention shows that the imaging frequency shifting concept improves the image quality in magnetic resonance imaging. The inventive method and apparatus provide fast magnetic resonance imaging of the heart and other organs to obtain high resolution and clear images.

Although modifications and changes may be suggested by those skilled in the art, it is the intention of the inventors to embody within the patent warranted hereon all changes and modifications as reasonably and properly come within the scope of their contribution to the art.

1. A method for reducing artifacts due to resonance frequency offsets in a diagnostic magnetic resonance (MR) image, comprising the steps of:
generating a plurality of MR scout images of a portion of a subject containing a region of interest (ROI) using respectively different radio frequency (RF) excitation frequencies, each of said MR scout images having an identifiable image quality in said ROI;
analyzing the plurality of MR scout images as to said image quality in the ROI and identifying one of said plurality of MR scout images having a best image quality in said ROI; and
generating a diagnostic MR image of said portion of said subject containing said ROI that is substantially free of artifacts due to resonance frequency offsets, using the RF excitation frequency used to generate said one of said plurality of MR scout images having said best image quality in the ROI.

2. A method as claimed in claim 1 comprising generating a plurality of MR coronary images as said plurality of MR scout images, and generating a diagnostic coronary MR image as said diagnostic MR image.

3. A method as claimed in claim 1 comprising generating a plurality of MR fat images as said plurality of MR scout images, and generating a diagnostic coronary MR image as said diagnostic MR image.

4. A method as claimed in claim 1 comprising generating a plurality of MR water images as said plurality of MR scout images, and generating a diagnostic coronary MR image as said diagnostic MR image.

5. A method as claimed in claim 1 comprising generating said diagnostic MR image using a true-FISP sequence.

6. A method as claimed in claim 1 comprising generating said plurality of MR scout images during a single breath-hold by said subject.

7. A method as claimed in claim 1 comprising generating said plurality of MR scout images of a single slice of said subject.

8. A method as claimed in claim 1 comprising generating said plurality of MR scout images with a low image resolution.

9. A method as claimed in claim 1 comprising generating said plurality of MR scout images of a single slice of said subject with a low resolution.

10. A magnetic resonance (MR) imaging apparatus comprising:
an MR scanner adapted to receive and interact with a subject therein, said MR scanner having a radio frequency (RF) resonator for exciting nuclear spins in the subject and for receiving MR signals resulting therefrom; and
a sequence controller connected to said MR scanner for operating said MR scanner, including operating said RF resonator, to generate a plurality of MR scout images of a portion of the subject containing a region of interest (ROI) using respectively different RF excitation frequencies, each of said MR scout images having an identifiable image quality in said ROI, and said sequence controller allowing analysis of said plurality of MR scout images as to said image quality in the ROI to identify one of said plurality of MR scout images having a best image quality in the ROI, and said sequence controller thereafter operating said MR scanner and to generate a diagnostic MR image of said portion of said subject containing said ROI, that is substantially free of artifacts due to resonance frequency offsets, using the RF excitation frequency used to generate said one of said plurality of MR scout images having said best image quality in the ROI.

11. An apparatus as claimed in claim 10 wherein said sequence controller automatically electronically identifies said one of said MR scout images having said best image quality in the ROI.

12. An apparatus as claimed in claim 10 comprising a display connected to said sequence controller at which each of said plurality of MR scout images is displayed for manual observation, and comprising an input unit allowing an operator to select, from among the displayed plurality of MR scout images, said one of said MR scout images having said best image quality in the ROI.

13. An apparatus as claimed in claim 10 wherein said sequence controller operates said MR scanner, including said RF resonator, in a true-FISP sequence for generating said diagnostic MR image.

14. An apparatus as claimed in claim 10 wherein said sequence controller operates said MR scanner, including said RF resonator, to generate said plurality of MR scout images of a single slice of the subject.

15. An apparatus as claimed in claim 10 wherein said sequence controller operates said MR scanner, including said RF resonator, to generate said plurality of MR scout images with a low resolution.

16. An apparatus as claimed in claim 10 wherein said sequence controller operates said MR scanner, including said RF resonator, to generate said plurality of MR scout images of a single slice of the subject with a low resolution.
